(19) United States
(12) Patent Application Publication
(10) Pub. No.: US 2010/0210824 A1
(43) Pub. Date: Aug. 19, 2010

(54) Method of Identifying Drugs, Targeting Moieties or Diagnostics

(76) Inventor: Jorn Gorlach, Manchester, NJ (US)

Correspondence Address: LICATA & TYRRELL P.C., 66 E. MAIN STREET, MARLTON, NJ, 08053, US

(21) Appl. No.: 12/769,036

(22) Filed: Apr. 28, 2010

Related U.S. Application Data
(63) Continuation of application No. 11/221,038, filed on Sep. 7, 2005.
(60) Provisional application No. 60/608,342, filed on Sep. 9, 2004.

(51) Int. Cl. C07K 16/00; C40B 30/04

(52) U.S. Cl. 530/387.1; 506/9

(57) ABSTRACT

The present invention relates to a method for identifying a binding agent or epitope for use in drug design, drug targeting or diagnostics. The method employs contacting and sorting binding agents and cognate epitopes from collections thereof, characterizing the binding agent and cognate epitope, detecting the level or location of the epitope in a sample using the binding agent, and correlating the level or location of the epitope in the sample with the presence or stage of a disease or condition to identify novel drugs, targeting moieties, or diagnostic agents.

This application is a continuation of U.S. Ser. No. 11/221,038 filed Sep. 7, 2005, which claims the benefit of U.S. Provisional Application No. 60/608,342, filed Sep. 9, 2004, each of which are herein incorporated by reference in their entireties.

### BACKGROUND OF THE INVENTION

Drugs currently marketed are directed at approximately 500 biological targets, almost exclusively of proteinaceous nature. Many of these targets are not fully understood and many of the drugs acting on them have significant side effects. Hence, there is a need for the identification of new, validated drug targets for subsequent development of novel therapeutics.

A single gene usually results in a collection of similar, but distinctly different groups of polypeptide products, due to RNA splicing, editing, maturation and multiple polypeptide processing steps. The individual polypeptide variants may have quite discrete biological functions and often only one specific variant of a family of polypeptides will be responsible for the main biological function encoded by the original gene. Gene-based approaches such as gene mapping, genetic transformation, gene knock-outs, and gene expression profiling used in the identification of new drug targets fail to detect molecular modifications downstream of RNA splicing and, thus, are not useful to investigate a majority of polypeptides in the human body. Moreover, there is no linear relationship between the number of genes in the human genome encoding a polypeptide family, the concentration of the corresponding mRNA, and the concentration of the resulting polypeptide. Thus, DNA and RNA-based technologies do not provide information on diseases that are manifested in the early steps in proteinogenesis.

Despite their specific role in disease, individual polypeptide variants play an important role in drug efficacy, absorption, distribution, metabolism and excretion. Most drugs developed with standard methods and enzyme-based screening assays are targeted toward a very specific individual variant of a given polypeptide. This polypeptide very often does not represent a human variant but an artificial form produced by bacteria, yeast, or mammalian cell lines. The resulting drugs are consequently specific inhibitors of that specific variant and may not be active against the critical polypeptide variant present in diseased patients. Consequently, these drugs typically fail in clinical trials. Given the breadth of polypeptide variation, drugs can have quite different effects on each individual patient. It is estimated that 50-60% of people taking a given drug receive the desired effect, while up to 5% have side effects and the remaining individuals receive no therapeutic effect.

Ultimately, it is desirable to investigate polypeptides, as opposed to the nucleic acids encoding them, to fully understand the origins of disease and develop the appropriate drugs. Common protein-based drug discovery technologies rely on mass spectrometry, two-dimensional polyacrylamide electrophoresis (2-D PAGE), and two-hybrid analysis. Mass spectroscopy and 2D-PAGE are relatively expensive and require expertise to obtain reproducible results. Moreover, mass spectroscopy and 2D-PAGE only provide structural information, but not functional properties. Functional information is indirectly provided by querying databases using the available structural data. Two-hybrid analysis does provide functional information but this technology is limited to proteins that can be expressed from a plasmid and typically excludes most cell surface proteins which are involved in signal transduction and cell-to-cell interactions. The most common two-hybrid system used is yeast. Unfortunately, yeast lacks the post-translational modification genes necessary for processing many human-related proteins. Therefore, functional binding experiments using the yeast two-hybrid system may not be optimal. Arrays of antibodies and proteins are described for use in drug discovery as well; see U.S. Pat. Nos. 6,329,209; 6,365,418; and 6,287,768; and WO 02/14866. Moreover, U.S. Patent Application No. 20020009740 discloses a metabolomics approach to discover small molecules associated with a disease state for disease treatment and diagnosis.

### SUMMARY OF THE INVENTION

The present invention is a method for identifying a binding agent or epitope for use in drug design, drug targeting or diagnostics. The method involves contacting a collection of binding agents with a collection of epitopes so that a cognate binding agent and epitope bind; sorting the bound binding agent and epitope from the collection; characterizing the binding agent and epitope; detecting the level or location of the characterized epitope in a sample using the characterized binding agent; and correlating the level or location of the epitope in the sample with the presence or stage of a disease or condition so that a binding agent or epitope for use in drug design, drug targeting or diagnostics is identified. In one embodiment, the steps of contacting a collection of binding agents with a collection of epitopes so that a cognate binding agent and epitope bind and sorting the bound binding agent and epitope from the collection occur simultaneously. In another embodiment, the method further includes the step of comparing the correlated level or location of the epitope in the sample with information in a database or publication.

### BRIEF DESCRIPTION OF THE DRAWINGS

FIG. 1 is a schematic showing the steps involved in carrying out the method of the present invention.

FIG. 2A shows human lung protein lysate, coupled to fluorescent beads, labeling the surface of a B-cell.

FIG. 2B shows the production of IgM antibodies by single, sorted B-cells after binding to cognate antigens from human lung fibroblasts.

FIG. 3 depicts particular embodiments for sorting and characterizing antigens having cognate binding partners. For example, antibodies can be immobilized on, e.g., beads for binding to cognate antigens, wherein upon sorting, the antigen is eluted and characterized via mass spectrometry (Panel A). Alternatively, the antibodies or antigens are immobilized on an array to bind the corresponding antigen or antibody, respectively (Panel B). Subsequently, the antigen is antigen is characterized via mass spectrometry.

### DETAILED DESCRIPTION OF THE INVENTION

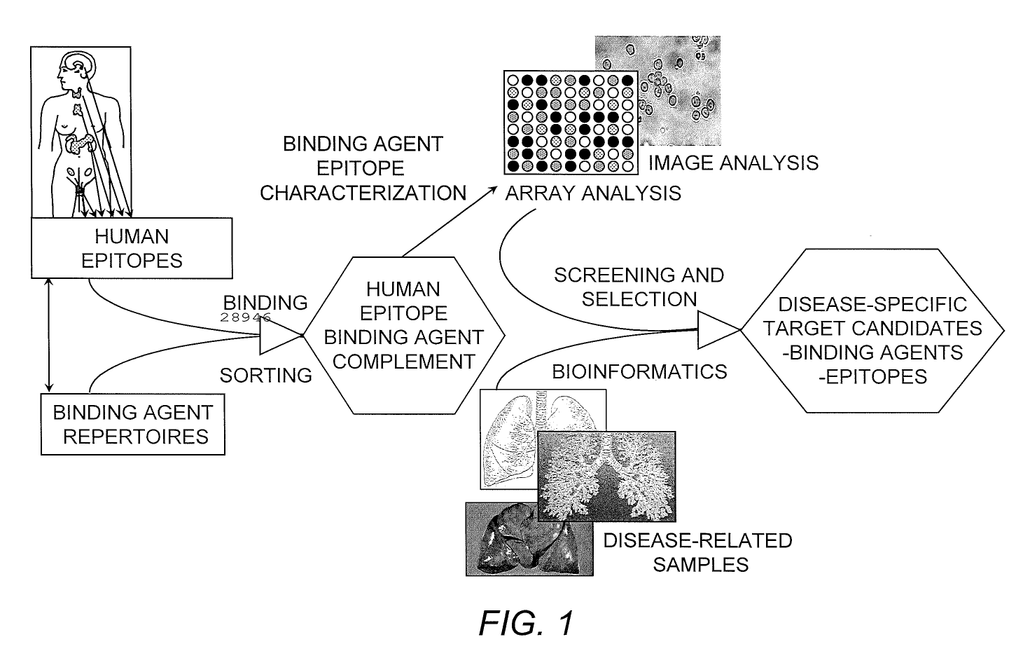

The present invention is an efficient, high throughput method for identifying binding agents or epitopes for use in drug design and drug targeting or diagnostics. The method employs the steps of contacting a collection of binding agents with a collection of epitopes so that a cognate binding agent and epitope bind; sorting the bound binding agent and epitope from the collection; characterizing the binding agent and epitope; detecting the level or location of the characterized epitope in a sample using the characterized binding agent; and correlating the level or location of the epitope in the sample with the presence or stage of a disease or condition (FIG. 1).

### I. Binding Agents and Epitopes

Within the scope of the invention, a binding agent is intended to include an antibody, an antibody fragment or derivative thereof, a peptide, an aptamer, or other non-protein based entity, such as a carbohydrate or lipid, which specifically binds to a cognate epitope. Such a carbohydrate or lipid may or may not be covalently attached to a protein (e.g., as a post-translational modification). In a particular embodiment of the present invention, the binding agent is an antibody, antibody fragment or derivative thereof. In the method of the invention, a binding agent specifically binds to its cognate epitope and can be used for sorting, characterizing, detecting, targeting, or localizing the epitope. In general, a collection of binding agents can be isolated from a sample (e.g., antibodies or peptides isolated from a sample of blood), can be generated in vitro (e.g., immunizing an animal with a collection of epitopes to generate a collection of cognate binding agents) or recombinantly- or chemically-synthesized (e.g., synthesizing a collection of peptides or antibodies).

An epitope, as used herein, is used in the broadest sense. Epitope is intended to include the classical definition, i.e., a portion of an antigenic macromolecule recognized and bound by a specific antibody, as well as any three-dimensional structure on a macromolecule which specifically interacts with a binding agent, e.g., a binding domain. By way of example, both a ligand mimetic anti-CD40 antibody and CD40 ligand would be considered binding agents which specifically bind to a CD40 epitope. While an epitope can be a protein or peptide, it can also be a carbohydrate, nucleic acid or lipid and is, in general, isolated from a sample prior to use in the instant method.

An epitope can be found on only one macromolecule or it can be found on two closely related macromolecules, e.g., homologs, orthologs, members of a protein family, isoforms, and the like. Desirably the epitope is found on fewer than five distinct macromolecules, more suitably two distinct macromolecules. In particular embodiments, the epitope is found on one macromolecule.

When a collection of epitopes or collection of binding agents is derived from a sample the collection can contain intracellular, extracellular, and/or secreted macromolecules of known or unknown identity or function. A collection of epitopes can be an extract from a whole sample or a fraction of the sample. Moreover, a collection of epitopes can be related macromolecules. The different epitopes can be either functionally related or suspected of being functionally related. The epitopes can share a similarity in structure or sequence or are suspected of sharing a similarity in structure or sequence. For instance, a collection of epitopes can be all growth factor receptors, hormone receptors, neurotransmitter receptors, catecholamine receptors, amino acid derivative receptors, cytokine receptors, extracellular matrix receptors, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors, cell-surface antigens, hepatitis C virus (HCV) proteases or HIV proteases, or polypeptides isolated from a specific cell, organ or tissue type. A collection of epitopes can be similar types of post-translation modifications, such as phosphorylated residues or O- or N-linked carbohydrates. Moreover, the collection of epitopes or collection of binding agents can be from a specific disease, physiological or developmental state.

As used herein, a disease or disease state or condition refers to any perturbation of the normal state that results in a change in epitope expression patterns or localization. Examples of perturbations include, but are not limited to, exposure to an allergen; immunological disorders; neoplasms; malignancies; metabolic disorders; all organ and tissue disorders, such as cardiac, liver, prostate, lung, pancreas, skin, eye, nervous system, lymphatic system, colon and breast disorders; aging; dementia; mental disorders; therapeutic drug treatment; and medical interventions, such as grafts, transplants, drug disorders, pathogen attack, or drought or saline growth conditions (e.g., in plants).

When a collection of binding agents or epitopes is isolated from a sample, the sample is generally of biological origin such as a cellular complex, organelle, cell, tissue, organ, bodily fluid or whole organism.

Cellular complexes include microtubules, ribosomes, cytoskeleton, cell wall, or cytosol which can be fractionated using well-known methodologies.

An organelle includes a nucleus, nucleolus, endoplasmic reticulum, Golgi apparatus, mitochondria, vacuole, peroxisome, lysosome or plastid. Gradient centrifugation and the like are well-known methods for isolating organelles from whole cells.

A cell includes, but is not limited to, a B-cell, T-cell, Helper T-cell, NK-cell, dendritic cell, macrophage, monocyte, neoplasm, white blood cell, red blood cell, muscle fiber, basal cell and nerve cell derived from the primary tissue of animals or from immortalized cell lines. Moreover, malignant tumor cells include those derived from a carcinoma, sarcoma or blastoma. Plant cells such as a mesophyll cell, sieve element, guard cell, epidermal cell are also considered cells of the present invention. Further, single cell organisms and specific cell types from lower multicellular organisms (e.g., spore or mycelia cells of fungi) are also contemplated.

A tissue includes connective, epithelial, muscle and nerve tissue from animals or parenchyma, collenchyma, sclerenchyma, xylem, phloem or epidermal tissue from plants.

An organ can be derived from the musculoskeletal system (e.g., muscles, bone and cartilage); the respiratory system (e.g., lungs); the digestive system (e.g., teeth, esophagus, stomach, small intestine and large intestine); the circulatory system (e.g., heart, capillaries, arteries and veins); the immune system (e.g., lymph nodes, bone marrow, spleen and thymus gland); the excretory system (e.g., kidneys, ureter, urethra and bladder); the nervous system (e.g., brain, ear, eye, spinal cord and nerves); the endocrine system (e.g., pituitary, pineal gland, hypothalamus, thymus, pancreas, thyroid and adrenals); the reproductive system (e.g., testis, ovaries, prostate gland and uterus); and the integument system (e.g., skin, hair and nails) derived from animals. Organs derived from plants include leaves, roots, stems, stamens, pistils and fruits.

A bodily fluid includes whole blood, plasma, serum, sputum, cerebrospinal fluid, pleural fluid, urine and the like.

Whole organisms are included in the present invention because the physiology and physiological state of individuals can be diverse. For example, individuals in a disease state or undergoing therapeutic treatment have a different physiological state than that of a healthy individual.

Any of the above-mentioned samples can be isolated from any source including plants, animals, fungi, bacteria, protozoa and preferably human.

Methods of isolating macromolecules from a sample for use as binding agents or epitopes in the method of the invention are well-known in the art. As one skilled in the art can appreciate, no one method may be applicable to all biological samples due to the nature of the biological sample, for example, extraction of macromolecules from bone can require different methodology than extraction of macromolecules from soft tissue. However, in all cases, the initial extraction technique must be compatible with downstream processing and experimentation with the ultimate objective of producing a sample which is soluble and maintains a native conformation.

Normalization of a collection can also be performed to remove macromolecules that are in high abundance relative to other macromolecules in the collection, for example, hemoglobin is an abundant protein in red blood cells, representing 95% of the total protein. Normalization can be performed using a plurality of adsorbents. Examples of adsorbents used in retentate chromatography are described in U.S. Pat. No. 6,225,047, herein referenced in its entirety.

Furthermore, a collection can be fractionated using liquid-phase fractionation techniques such as chromatography (Labrou (2003) J. Chromatogr. B Analyt. Technol. Biomed. Life Sci. 790(1-2):67-78), hydrophobic, hydrophilic, isoelectric focusing, ligand binding, and size separation. Systems used to achieve such separations include, but are not limited to, High-Performance Liquid Chromatography (HPLC), Fast Protein Liquid Chromatography (FPLC), capillary electrophoresis and reverse-phase HPLC. Depending on buffer conditions, sample size, and concentration of the fractionated macromolecules, samples can be further concentrated or desalted using methods such as trichloroacetic acid, acetone, and ammonium sulfate precipitation or vacuum evaporation prior to the next step.

Collections of peptides and polypeptides can be still further separated using PAGE separation methodologies. One such methodology is two-dimensional PAGE (2-D PAGE) (see, e.g., O'Farrell (1975) J. Biol. Chem. 250(10):4007-21; O'Farrell and O'Farrell (1977) Methods Cell Biol. 16:407-20; and O'Farrell, et al. (1977) Cell 12(4):1133-41). A plurality of electrophoretic conditions can be used to optimize separation of any given peptide or polypeptide sample. For example, electrophoretic conditions may be Non-Equilibrium pH Gradient Electrophoresis (NEPHGE) or Isoelectric Focusing (IEF) and a plurality of ampholine concentrations may be employed.

While a collection of binding agents containing antibodies or antibody fragments can be obtained from a sample, a collection of antibodies or antibody fragments can also be produced by natural (i.e., immunization) or partial or wholly synthetic means. All derivatives thereof which maintain specific binding ability are also included. Antibodies cam be monoclonal or polyclonal and include commercially available antibodies, against known, well-characterized polypeptides. An antibody can be a member of any immunoglobulin class, including any of the human classes: IgG, IgM, IgA, IgD, and IgE. Derivatives of the IgG class, however, are desirable. Further, an antibody can be of human, mouse, rat, goat, sheep, rabbit, chicken, camel, or donkey origin or other species which may be used to produce native or human antibodies (i.e, recombinant bacteria, baculovirus or plants).

Antibody fragments can be any derivative of an antibody which is less than full-length. Desirably, an antibody fragment retains at least a significant portion of the full-length antibody's specific binding ability. Examples of antibody fragments include, but are not limited to, Fab, Fab′, F(ab′)₂, scFv, Fv, dsFv, diabody, Fd fragments or microbodies, for example, U.S. Patent Application No. 20020012909. The antibody fragment can be produced by any means. For instance, the antibody fragment can be enzymatically or chemically produced by fragmentation of an intact antibody or it can be recombinantly-produced from a gene encoding the partial antibody sequence. As used herein, antibody also includes bispecific and chimeric antibodies.

Alternatively, the antibody fragment can be wholly or partially synthetically-produced. An antibody fragment can optionally be a single-chain antibody fragment. Alternatively, a fragment can contain multiple chains which are linked together, for instance, by disulfide linkages. A fragment can also optionally be a multi-molecular complex. A functional antibody fragment will typically include at least about 50 amino acids and more typically will include at least about 200 amino acids.

Naturally-produced monoclonal antibodies can be generated using classical cloning and cell fusion techniques or techniques wherein B-cells are captured and nucleic acids encoding a specific antibody are amplified. Whole sample extracts, a fraction thereof or an individual peptide or polypeptide can be used for the initial immunization and in the context of antibody production is referred to herein as the antigen. In one embodiment, the antigen is a total sample extract or a fraction thereof to generate a large pool of uncharacterized antibodies. The antigen of interest is typically administered (e.g., intraperitoneal injection) to wild-type or inbred mice (e.g., BALB/c) or rats, rabbits, chickens, sheep, goats, or other animal species which can produce native or human antibodies. The antigen can be administered alone, or mixed with adjuvant. After the animal is boosted, for example, two or more times, the spleen or large lymph node, such as the popliteal in rat, is removed and splenocytes or lymphocytes are isolated and fused with myeloma cells using well-known processes, for example, see Kohler and Milstein ((1975) Nature 256:495-497) or Harlow and Lane (Antibodies: A Laboratory Manual (Cold Spring Harbor Laboratory, New York (1988)). The resulting hybrid cells are then cloned in the conventional manner, e.g. using limiting dilution, and the resulting clones, which produce the desired monoclonal antibodies, are cultured (see Stewart, S. (2001) Monoclonal Antibody Production. In: Basic Methods in Antibody Production and Characterization. (Howard and Bethell (eds.), CRC Press, Boca Raton, Fla., pp. 51-67).

Alternatively, antibodies can be derived by a phage display method. Methods of producing phage display antibodies are well-known in the art, e.g., see Huse, et al. ((1989) Science 246(4935):1275-81). Selection of antibodies is based on binding affinity to epitopes from a sample extract or a fraction thereof. In this embodiment, some or many of the antibodies bind peptides or polypeptides of unknown identity and/or function.

Recombinant production of a collection of binding agents which contain proteins or peptides can require isolation of a collection of nucleic acid sequences from a sample and incorporation into a recombinant expression vector in a form suitable for expression of the collection of proteins or peptides in a host cell. A suitable form for expression provides that the recombinant expression vector includes one or more regulatory sequences operatively-linked to the nucleic acids encoding the collection of proteins or peptides in a manner which allows for transcription of the nucleic acids into mRNA and translation of the mRNA into the proteins or peptides. Regulatory sequences can include promoters, enhancers and other expression control elements (e.g., polyadenylation signals). Such regulatory sequences are known to those skilled in the art and are described in Goeddel D. D., ed., Gene Expression Technology, Academic Press, San Diego, Calif. (1991). It should be understood that the design of the expression vector may depend on such factors as the choice of the host cell to be transfected and/or the level of expression required. Nucleic acid sequences or expression vectors harboring nucleic acid sequences encoding a collection of proteins or peptides may be introduced into a host cell, which may be of eukaryotic or prokaryotic origin, by standard techniques for transforming cells. Suitable methods for transforming host cells may be found in Sambrook, et al. (Molecular Cloning: A Laboratory Manual, 3rd Edition, Cold Spring Harbor Laboratory Press (2000)) and other laboratory manuals. The number of host cells transformed with nucleic acid sequences encoding a collection of proteins or peptides will depend, at least in part, upon the type of recombinant expression vector used and the type of transformation technique used. Nucleic acids can be introduced into a host cell transiently, or more typically, for long-term expression of a collection of proteins or peptides, the nucleic acid sequences are stably integrated into the genome of the host cell or remain as a stable episome in the host cell. Once produced, a collection of proteins or peptides can be recovered from culture medium as secreted polypeptides or peptides, although it also can be recovered from host cell lysates when directly expressed without a secretory signal. When a collection of proteins or peptides is expressed in a recombinant cell other than one of human origin, the collection of proteins or peptides is substantially free of proteins or polypeptides of human origin.

In addition to recombinant production, a collection of proteins or peptides, antibodies, lipids or carbohydrates can be produced using solid-phase techniques (see, e.g., Merrifield J. (1963) J. Am. Chem. Soc. 85:2149-2154; Seeberger (2003) Chem. Commun. (Camb) (10):1115-21). Protein synthesis can be performed using manual techniques or by automation. Automated synthesis may be achieved, for example, using Applied Biosystems 431A Peptide Synthesizer (Perkin Elmer, Boston, Mass.). Various peptides or fragments of proteins of a collection of proteins can be chemically-synthesized separately and combined using chemical methods to produce a full-length molecule.

Further combinatorial chemistry approaches can be used to produce collections of epitopes or binding agents (see, e.g., Lenssen, et al. (2002) Chembiochem. 3(9):852-8; Khersonsky, et al. (2003) Curr. Top. Med. Chem. 3(6):617-43; Anthony-Cahill and Magliery (2002) Curr. Pharm. Biotechnol. 3(4):299-315).

A collection, as defined herein, is intended to be more than one distinct binding agent and more than one distinct epitope, generally between about 5 and 1000 or more suitably between about 100 and 10,000. In particular embodiments, a plurality is between about 1000 and 100,000. A collection can also be more than 100,000 or more than one million.

### II. Contacting and Sorting

In general, the step of contacting a collection of binding agents with a collection of epitopes will be maintained for a sufficient period of time for binding between the binding agent and epitope binding partner to occur.

In one embodiment, individual epitopes of a collection or individual binding agents of a collection can be placed in a well or spot on a membrane (i.e., in an array) and contacted with a collection of binding agents or collection of epitopes, respectively, so that individual binding agents and their cognate epitopes bind. When either the collection of epitopes or collection of binding agents are separated on an array prior to contact with the cognate binding partner, the step of sorting the bound binding agent and epitope from the collection occurs simultaneously with the contacting step.

Methods of arraying macromolecules are well-known in the art. Typically, arrays comprise micrometer-scale, two-dimensional patterns of patches of macromolecules (i.e., binding agents or epitopes) immobilized on an organic thin-film coating on the surface of the substrate. Examples of arrayed macromolecule chips, including array pattern and density, substrates, coatings and organic thin-films are described in the art, for example, WO 02/14866; U.S. Pat. Nos. 6,329,209; and 6,365,418, each of which are incorporated by reference in their entirety.

An array of macromolecules comprises a substrate, at least one organic thin-film covering some or all of the surface of the substrate, and a plurality of patches arranged in discrete, known regions on the portions of the substrate surface covered by organic thin-film, wherein each patch comprises macromolecules immobilized on the organic thin-film, wherein said macromolecules of a given patch binds a particular cognate binding partner in a collection, and the array comprises a plurality of macromolecules, generally between about 10 and 10,000, each of which binds a different cognate binding partner in a collection.

The macromolecules are preferably covalently immobilized on the patches of the array, either directly or indirectly, for example, protein A may be used to orient an antibody with the binding region above the substrate surface.

In general, only one type of macromolecule is present on a single patch of the array. If more than one type of macromolecule is present on a single patch, all of the macromolecules of that patch must share a common binding partner. For example, a patch can contain a variety of antibodies to the same polypeptide although, potentially, the antibodies can bind different epitopes on that same polypeptide.

Optimal binding is achieved by contacting a plurality of binding agents or epitopes on an array with a plurality of cognate binding partners in a suitable container, under a cover slip, etc, or by incorporation into a structure that provides ease of analysis, high throughput, or other advantages, such as in a biochip format, a multiwell format and the like. For example, the subject arrays could be incorporated into a biochip type device. A biochip device is, for example, a substantially rectangular shaped cartridge containing fluid entry and exit ports and a space bounded on the top and bottom by substantially planar rectangular surfaces, wherein the array is present on one of the top and bottom surfaces. Such a device is disclosed in U.S. Pat. No. 6,287,768 and is incorporated herein by reference in its entirety.

Alternatively, the subject arrays could be incorporated into a high throughput or multiwell device, wherein each array is bounded by raised walls in a manner sufficient to form a reaction container wherein the array is the bottom surface of the container.

Contact of an array and a plurality of binding partners involves contacting the array with an aqueous medium containing the binding partners. Contact can be achieved in a variety of different ways depending on specific configuration of the array. For example, where the array is incorporated into a biochip device having fluid entry and exit ports, the probe solution can be introduced into the chamber in which the pattern of target molecules is presented through the entry port, where fluid introduction could be performed manually or with an automated device. In multiwell embodiments, the probe solution will be introduced in the reaction chamber containing the array, either manually, e.g., with a pipette, or with an automated fluid handling device. Alternatively, the array can be subjected to centrifugal force to overcome non-specific binding forces that limit the rate of liquid flow, thus allowing for an increase in agitation and related replenishment rates. Such an apparatus used to facilitate array hybridization is disclosed in U.S. Pat. No. 6,309,875, which is incorporated herein by reference in its entirety.

In an alternative embodiment, the collection of binding agents and collection of epitopes are contacted prior to the step of sorting by adding the collection of binding agents to a point of application, such as a tube or a well in a plate containing the collection of epitopes so that individual binding agents and their cognate epitopes bind. Subsequently, the bound binding agents and cognate epitopes are sorted from other bound and non-bound members of the collections. In this embodiment, the step of sorting is generally carried out using cell-sorting methods such as fluorescence-activated cell sorting (FACS), hydraulic or laser capture microdissection in combination with laser confocal microscopy or fluorescence microscopy, or changes in mass. For convenience, the epitope and/or the binding agent can be presented on the surface of a cell or phage, contacted with the cognate binding partner and sorted based on the binding interaction. Alternatively, a collection of immobilized binding agents (e.g., immobilized on magnetic beads) can be contacted with a collection of free epitopes, allowed to bind, and separated based on the binding interaction. As a further alternative, a collection of immobilized epitopes can be contacted with a collection of free binding agents, allowed to bind, and separated based on the binding interaction. While no label may be used in the step of sorting bound binding agents and epitopes, typically, either one or both (i.e., applying Fluorescence Resonance Energy Transfer (FRET) or bioluminescence resonance energy transfer (BRET) techniques) binding partners are labeled, preferably with a fluorescent or bioluminescent tag, and upon binding are detected and sorted based on the binding interaction. Fluorochromes such as Phycocyanine, Allophycocyanine, Tricolor, AMCA, Eosin, Erythrosin, Fluorescein, Fluorescein Isothiocyanate Hydroxycoumarin, Rhodamine, Texas Red, Lucifer Yellow, and the like may be attached directly to one or both binding partners through standard groups such as sulfhydryl or primary amine groups. Those of ordinary skill in the art will know of other suitable labels which can be employed in accordance with the present invention. The binding of these labels to antibodies or fragments thereof can be accomplished using standard techniques (see, for example, Kennedy, et al. (1976) Clin. Chim. Acta 70:1-31 and Schurs, et al. (1977) Clin. Chim Acta 81:1-40).

Presentation of a binding agent or epitope on a cell surface may be accomplished using standard methods such as yeast display (see, Feldhaus, et al. (2003) Nat. Biotechnol. 21(2):163-70), E. coli display (see, Kjaergaard, et al. (2002) J. Bacteriol. 184(15):4197-204; Alcala, et al. (2003) FEBS Lett. 533(1-3):115-8) or display on any cell that can be transfected to present the binding agent or epitope on the cell surface, (e.g., B cells). By way of illustration, a collection of antibodies (i.e., binding agents) can be presented on the surface of a yeast cell or B-cell isolated from a sample; mixed with a collection of fluorescently-labeled proteins (i.e., epitopes) isolated from a cancer cell sample so that the cognate binding partners interact and bind; and fluorescently-labeled cells, which represent interacting binding partners, are sorted by FACS into individual wells of a microtiter plate.

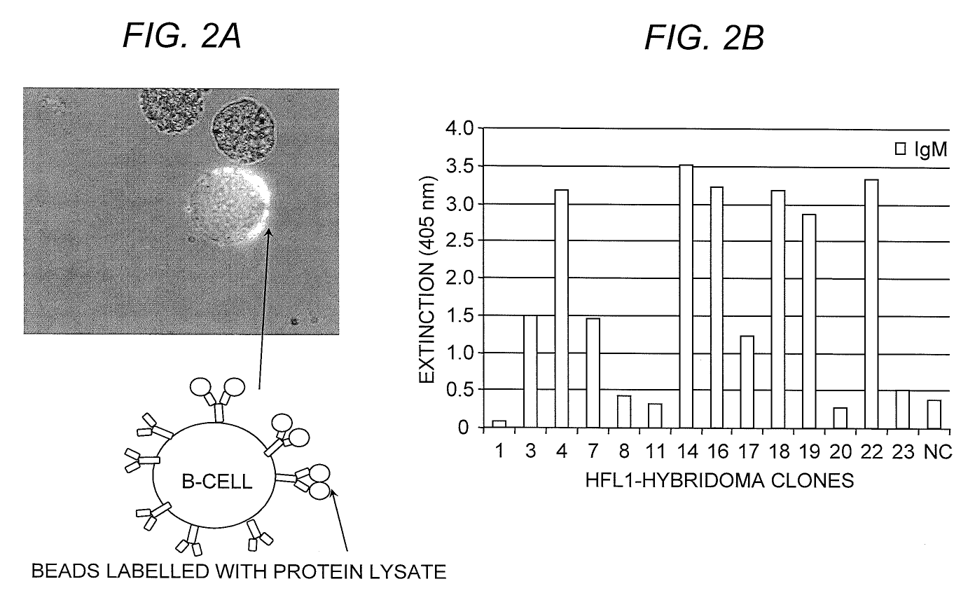

Using the binding and sorting steps of the present invention, single, sorted B-cells were isolated which produced IgM antibodies specific for human lung proteins (FIG. 2).

### III. Characterizing

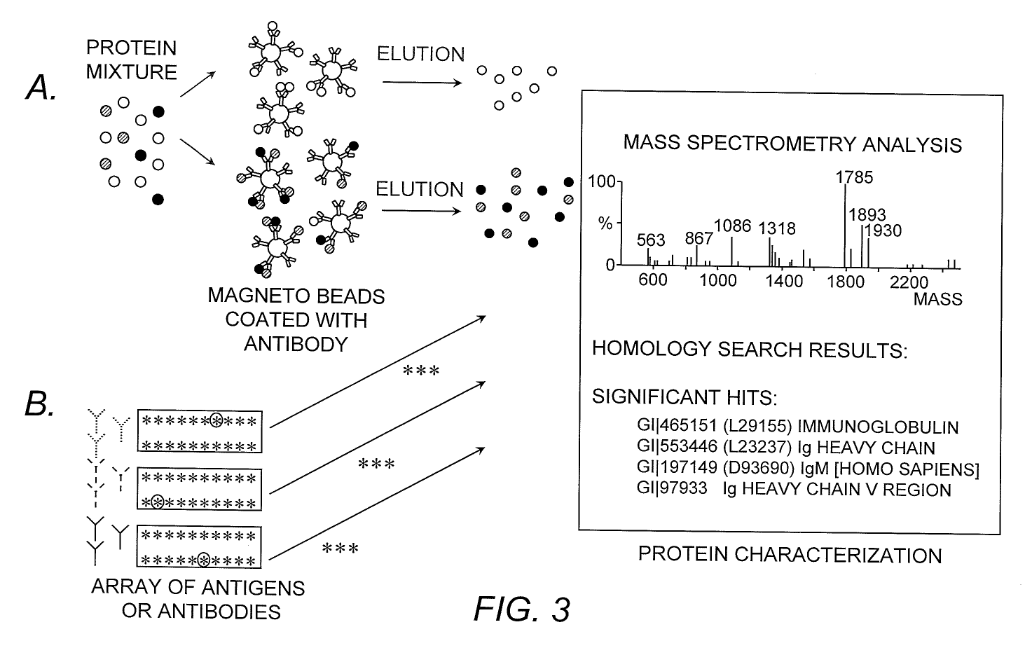

Bound and sorted binding partners are subsequently characterized. The sorted binding agent and cognate epitope can be separated from one another and individually characterized or characterized as a bound entity. For example, if the binding agent is an antibody and the epitope is a protein or peptide, the antibody can remain bound to the microtiter plate well or beads and the protein eluted for direct mass spectroscopy analysis (FIG. 3). Characterization of a binding agent or epitope includes determining the physical properties such as sequence (e.g., amino acid sequence of a protein or nucleic acid sequence encoding an antibody presented on a B-cell), structure (including primary, secondary or tertiary structure), activity or mass.

When an epitope or binding agent is presented on the surface of a cell, the sequences flanking the nucleic acid sequences encoding the binding agent or epitope are preferably known. In this manner, using such methods as single-cell PCR (Coronella, et al. (2000) Nucleic Acids Res. 28(20):E85) and automated DNA sequencing, the nucleic acid sequences encoding the epitope or binding agent can be determined. For example, when the binding agent is an antibody presented on the surface of a yeast cell, the heavy and light chain antibody domains can be amplified by PCR using antibody-specific oligonucleotides (see, e.g., Sblattero and Bradbury (1998) Immunotechnology 3: 271-278) and characterized by sequencing. Alternatively, the amplicons can be cloned into an expression vector and expressed in a host cell to produce large quantities for further characterization.

When the quantities of only one of the binding Partners is sufficient for further characterization, a second binding step or matching step can be employed to obtain information on the other, low quantity binding partner. For example, if the binding agent is an antibody presented on a yeast cell (wherein the nucleic acid sequences encoding the antibody are isolated and can be expressed to produce large quantities of the select antibody) and the epitope is one or two molecules of an individual protein, the collection of epitopes from which the protein was originally sorted can be concentrated, fractionated and/or separated on a 2-D gel and contacted with the amplified antibody to identify the cognate isolated protein. Desirably, the separated proteins are transferred to a solid matrix (i.e. western blotted) and subsequently contacted with the amplified antibody to identify the cognate isolated protein. Thereafter, the identified cognate protein, present in greater quantities can be excised from the gel or solid matrix and analyzed by methods such as mass spectroscopy.

Western blotting techniques are well-known in the art of protein biochemistry. Proteins or peptides can be transferred to membranes such as polyvinylidene difluoride or other membranes or matrices (see, e.g., Strupat, et al. (1994) Anal. Chem. 66:464) and Vestling and Fenselau (1994) Anal. Chem. 66:471) using standard electrophoretic transport methods, e.g., Towbin, et al. ((1979) Proc. Natl. Acad. Sci. USA 76(9):4350-4).

In preparation for mass spectroscopy analysis, individual peptide or polypeptide spots on PAGE gels or solid matrices are excised and are subjected to fragmentation by a plurality of enzymes (e.g., trypsin) or chemicals (e.g., hydrochloric acid) well-known in the art, for example, U.S. Pat. No. 5,595,636, herein referenced in its entirety.

Peptide fragments are analyzed for mass and/or amino acid sequence determination using a plurality of mass spectroscopy (MS) methodologies well-known to one skilled in the art. For example, Matrix-Assisted Laser Desorption/Ionization-Time of Flight (MALDI-TOF), electrospray ionization liquid chromatography-MS/MS-TOF (ESI LC-MS/MS-TOF), MALDI MS/MS-TOF, ion-trap MS/MS, MALDI MS/MS-TOF-TOF or any combination of these methodologies may be employed.

Characterization also includes the identification of a plurality of binding agents which interact with an epitope or a plurality of epitopes which interact with a binding agent. For instance, if the epitope is of a protein which may have more than one epitope, there may be a plurality of binding agents which bind to said protein at the other epitopes. Such characterization can be carried out by, for example, contacting an array of a collection of characterized epitopes with a single binding agent to determine the single binding agent interacts with more than one characterized epitope. Likewise, a collection of characterized binding agents can be placed in an array and contacted with a single epitope to identify a plurality of binding agents which interact with the epitope. It is contemplated that one or more unique binding agent may exist for each epitope; hence, one or more patches on the array of binding agents will bind the same epitope. This property provides that each epitope can be bound to an array of binding agents in a plurality of conformations. Each conformation allows for unique binding interactions to occur with other molecular species.

Interactions between binding partners on an array can be visualized or detected using a plurality of methods including, but not limited to, non-labeled detection methods such as, surface plasmon resonance (SPR; Biacore International, AB, Uppsala, Sweden), planar waveguide (Zeptosens, Witterswil, Switzerland), surface enhanced laser desorption ionization (SELDI; Ciphergen Biosystems, Inc., Fremont, Calif.), and the like. Alternatively, visualization can be performed by labeling the epitope or binding agent with a variety of labels such as, fluorescent dyes, chemiluminescent markers, or bio-luminescent markers. To be effective, methods in which a label is used are reliant upon a consistent and uniform labeling technique across a vast mixture of epitopes or binding agents. Methods for labeling peptides or polypeptides either target a specific amino acid or target a number of known or unknown moieties, for example, glutaraldehyde.

### IV. Detecting

In accordance with the method of the invention, the step of detecting the level or location of the characterized epitope in a sample is carried out using its cognate, characterized binding agent.

This step of the method is intended to detect and measure the temporal or spatial expression of an epitope in a sample. A sample can be frozen, a live cell, sectioned, or fractionated by component (e.g., separation of carbohydrates from lipids and proteins) and/or arrayed. When determining the level of an epitope in a sample, desirably, the epitopes are cell-free extracts of a sample.

It is contemplated that the cognate binding agent is labeled with a fluorescent dye, chemiluminescent marker, bio-luminescent marker, or biotin to visualize and measure the level or location of the epitope in a sample. The time required for binding labeled binding agent with its cognate epitope can vary with temperature, extent of permeabilization of a cell, or sample or cell type. Additional reagents can be added to the medium containing the sample to decrease non-specific binding interactions or improve the stability of the binding partner interaction, e.g., bovine serum albumin or other reagents known to have such properties. Subsequently, the sample can be washed to remove any residual or non-bound labeled binding agent prior to visualization and analysis. Methods of visualizing and analyzing any of the above-mentioned labels are well-known in the art and the method employed will vary with the type of analysis being conducted, i.e. individual samples or multiple sample analyses in high-throughput screens. Desirably, measurement of the labeled binding partners is accomplished using flow cytometry, laser confocal microscopy, spectrofluorometer, fluorescence microscopy, immunocytochemistry, western blotting, ELISA, fluorescence scanners, electron microscopy and the like.

It is contemplated that detecting the level or generating an expression profile of an epitope is preferably conducted in an array format. An expression pattern is generated when one, two or a collection of epitopes from two or more samples are sequentially hybridized to the same array of one, two or a collection of binding agents to reveal differences or similarities in expression for each epitope between the samples.

An array of binding agents can be used to compare epitope expression patterns derived from a normal sample and samples form various stages of a disease state or condition to identify drug targets. Differences in expression patterns between the normal and stages of the disease state or condition will provide disease biomarkers, which may or may not be specific for said stage of a disease state, and in a particular embodiment can be used as drug targets or to diagnose the presence or stage of a disease state.

Localization or spatial expression of an epitope is desirably conducted on whole cells. The whole cells can be derived from a first sample and contacted with an array of binding agents to determine if the epitope is expressed on the cell surface. Cell surface epitopes such as carbohydrates, lipids or proteins can interact with the array of binding agents to provide the binding interaction.

Alternatively, the subcellular localization of an epitope can be detected by fractionating a cell into its individual organelles and applying whole organelles or organelle extracts to an array of binding agents to detect organelle-specific epitopes. Further, microscopic analysis of whole cell sections can be conducted to localize an epitope. For example, it is contemplated that changes in epitope localization can occur in a disease state or condition as compared to a normal state (e.g., a transcription factor which is no longer transported to the nucleus may contribute to loss of gene regulation which results in a disease state). Furthermore, it is contemplated that the structure and location of a macromolecule can be evaluated by comparing the binding of one or more binding agents known to bind to the same macromolecule, as determined during the characterization step of the invention. By way of illustration, a first antibody may recognize the phosphorylated, nuclear-localized isoform of a kinase whereas a second antibody may recognize the unphosphorylated, cytoplasmic-localized isoform of a kinase. Mutations in the kinase which contribute to a disease state may result in a loss of phosphorylation of the kinase which can be detected by loss of binding of first antibody in the nucleus.

### V. Correlating

The step of correlating the level or location of the epitope in the sample with the presence or stage of disease or condition is carried out by creating epitope expression or localization profiles of disease states as compared to normal to provide a plurality of disease biomarkers. Disease biomarkers are macromolecules that are absent, present, or whose expression or location is either modified or altered (e.g., an increase or decrease in expression) in the disease state as compared to the normal state. Disease biomarkers can be directly or indirectly involved in the manifestation of the disease state.

Epitopes found to be suitable biomarkers and the binding agents which interact with said epitope are suitable both as therapeutic and prophylactic agents for treating or preventing a disease state. The epitope or binding agent of interest can be used to design novel drugs, used in drug targeting or used for diagnostic purposes.

It is contemplated that the binding agent itself can be used as a drug or can be used in the design and synthesis of either peptide or non-peptide compounds (mimetics) specific to the epitope (see, e.g., Saragovi, et al (1991) Science 253:792-795) to alter the function or activity of epitope thereby altering the disease state or condition.

When the binding agent is an antibody not of human origin (i.e., produced by immunizing a mouse) it can be used for the production of humanized and chimeric antibodies, wherein the mouse antibody genes are spliced to human antibody genes to obtain a molecule with appropriate antigen specificity and biological activity (Morrison, et al. (1984) Proc. Natl. Acad. Sci. 81, 6851-6855; Neuberger, et al. (1984) Nature 312:604-608; Takeda, et al. (1985) Nature 314:452-454). Alternatively, techniques described for the production of single chain antibodies may be adapted, using methods known in the art. Antibodies with related specificity, but of distinct idiotypic composition, may be generated by chain shuffling from random combinatorial immunoglobulin libraries (Burton (1991) Proc. Natl. Acad. Sci. 88:11120-11123).

Anti-idiotype antibodies (Ab2) and anti-anti-idiotype antibodies (Ab3) can also be produced when the binding agent is an antibody. Ab2 are specific for the epitope to which the primary antibodies of the invention bind and Ab3 are similar to primary antibodies (Ab1) in their binding specificities and biological activities (see, e.g., Wettendorff, et al., “Modulation of anti-tumor immunity by anti-idiotypic antibodies.” In: Idiotypic Network and Diseases, ed. by J. Cerny and J. Hiernaux J, Am. Soc. Microbiol., Washington D.C.: pp. 203-229, (1990)). These anti-idiotype and anti-anti-idiotype antibodies may be produced using techniques well-known to those of skill in the art.

An epitope identified by the method of the invention may be used to identify an agent which binds to the epitope to alter its structure, function or activity. Cell-based and cell-free methods of screening a library of test agents are well-known in to the skilled artisan. Cell-free assays may comprise contacting purified epitope with a library of test agents and detecting binding between the test agent and epitope. Wherein the activity of the epitope is known, activity-based assays may be performed to evaluate whether the activity of an epitope is altered in the presence of a test agent. Libraries of test agents may comprise either collections of pure agents or collections of agent mixtures. Examples of pure agents include, but are not limited to, proteins, polypeptides, peptides, nucleic acids, oligonucleotides, carbohydrates, lipids, synthetic or semi-synthetic chemicals, and purified natural products. Examples of agent mixtures include, but are not limited to, extracts of prokaryotic or eukaryotic cells and tissues, as well as fermentation broths and cell or tissue culture supernatants. In the case of agent mixtures, the methods of this invention are not only used to identify those crude mixtures that possess the desired activity, but also provide the means to monitor purification of the active principle from the mixture for characterization and development as a therapeutic drug. In particular, the mixture so identified may be sequentially fractionated by methods commonly known to those skilled in the art which may include, but are not limited to, precipitation, centrifugation, filtration, ultrafiltration, selective digestion, extraction, chromatography, electrophoresis or complex formation. Each resulting subtraction may be assayed for the desired activity using the original assay until a pure, biologically active agent is obtained.

Library screening may be performed in any format that allows rapid preparation and processing of multiple reactions such as in, for example, multi-well plates of the 96-well variety. Stock solutions of the agents as well as cell lines and assay components are prepared manually and all subsequent pipetting, diluting, mixing, washing, incubating, sample readout and data collecting is done using commercially available robotic pipetting equipment, automated work stations, and analytical instruments for detecting the signal generated by the assay. Examples of such detectors include, but are not limited to, luminometers, spectrophotomers, calorimeters, and fluorimeters, and devices that measure the decay of radioisotopes.

A binding agent interacting with an epitope found to be involved in a disease state or condition may also be used as targeting moiety. A targeting moiety is defined as an agent which specifically targets a drug to a diseased cell of interest, preferably, the targeted epitope is localized on the cell-surface, and the cognate binding agent facilitates uptake of the drug into the cell of interest for treatment of the phenotypes associated with the disease state of the diseased cell.

For diagnostic purposes, binding agents which are antibodies or antibody fragments are desirable. An antibody or antibody fragment may be conjugated to a solid support suitable for a diagnostic assay (e.g., beads, plates, slides or wells formed from materials such as latex or polystyrene) in accordance with known techniques, such as precipitation. Antibodies may likewise be conjugated to detectable groups such as radiolabels (e.g., ³⁵S, ¹²⁵I, ¹³¹I) enzyme labels (e.g., horseradish peroxidase, alkaline phosphatase), and fluorescent labels (e.g., fluorescein) in accordance with known techniques.

Methods for detecting or diagnosing a disease state or condition or the risk of developing a disease state or condition using antibodies are well-known in the art. These methods typically rely on detecting the level or presence of an epitope associated with a disease state or condition in a sample and comparing said level or presence in the sample to a level or presence in a control. Once non-specific interactions are removed by, for example, washing the sample, the epitope-antibody complex is detected using any one of the well-known immunoassays used to detect and/or quantitate antigens. Exemplary immunoassays which may be used in the methods of the invention include, but are not limited to, enzyme-linked immunosorbent, immunodiffusion, chemiluminescent, immunofluorescent, immunohistochemical, radioimmunoassay, agglutination, complement fixation, immunoelectrophoresis, western blots, mass spectrometry, antibody array, and immunoprecipitation assays and the like which may be performed in vitro, in vivo or in situ. Such standard techniques are well-known to those of skill in the art (see, e.g., “Methods in Immunodiagnosis”, 2nd Edition, Rose and Bigazzi, eds. John Wiley & Sons, 1980; Campbell et al., “Methods and Immunology”, W.A. Benjamin, Inc., 1964; and Oellerich, M. (1984) J. Clin. Chem. Clin. Biochem. 22:895-904; Harlow and Lane, Antibodies: A Laboratory Manual, Cold Spring Harbor Laboratory, New York (1988) 555-612).

### VI. Comparing

While binding agents and epitopes for use in drug design, drug targeting, or diagnostics may be identified using the method of the present invention, it may be desirable to compare the correlated level or location of an epitope in a sample with information pertaining to the epitope available in a database or publication.

In accordance with the method of the invention, each epitope will be characterized to identify its mass, amino acid sequence, structure, function, expression patterns in any given disease state or developmental stage, location, isoforms, corresponding binding agent, protein interactions with other molecular species, and enzyme or metabolic pathway association. Data for each epitope is collected at a plurality of steps of the method disclosed herein and may be compared to data existing in known databases or publications. For example, locations of proteins in 2-D gels or matrices may be compared to data in the Protein Disease Database (PPD) (Merrill, et al. (1995) Appl. Theor. Electrophor. 5:49-54). Furthermore, mass and amino acid sequence data collected from MS analysis of each epitope may be compared to databases such as PPD, SwissProt, Protein Data Bank (PDB), GenPept, Ludwignr, NCBInr, Owl, Database of Proton NMR Spectra of Xyloglucans, SWEET-DB (http://www.dkfz.de/spec2/sweetdb/), LIPIDAT, and the like. Data acquisition and cataloguing are known to the art, for example U.S. Patent Application No. 20020028005.

Moreover, protein-protein interactions, protein structure, and enzyme and metabolic pathway data may be obtained from the scientific literature using automated extraction protocols, for example Ono, et al. ((2001) Bioinformatics 17(2):155-61) and Humphreys, et al. ((2000) Pac. Symp. Biocomput. 505-16).

### VII. Uses of Binding Agents and Epitopes Identified by the Method of the Invention

Binding agents and epitopes may be directly used in drug design, drug targeting, or diagnostics as described herein. Furthermore, arrays of binding agents may be used to profile epitopes derived from patient tissue samples at various intervals of drug treatment to identify epitopes that are regulated by said drug treatment. Furthermore, regulation of epitopes expression by drug candidates may be evaluated with model systems to determine drug toxicity and efficacy. For example, using an array of binding agents, profiles of epitope expression may be generated for samples treated with known therapeutic agents or known toxins. This may be accomplished with cell lines in vitro or in various model systems, depending on the disease state being investigated. These profiles are then compared to epitope expression profiles of samples treated with unknown agents or toxins. As more profiles are generated, more definitive information concerning unknown agents or toxins is elucidated. In addition, these same profiles may be compared against patient profiles to monitor efficacy and toxicity of therapeutic drug treatment. This may provide valuable information at all stages of clinical drug trials as well as subsequent monitoring of patients undergoing drug treatment.

Furthermore, an array of binding agents may be used in a clinical or hospital setting to identify patients that may have an adverse reaction to a specific drug or class of drugs or that might react in a very positive manner to a certain therapeutic drug treatment. A patient tissue sample would be taken and analyzed by the appropriate array of binding agents to produce a disease biomarker profile. The profile may be generated at one time point or over multiple time points. These profiles are then compared to a vast database of profiles from other patients, treatments, model systems, and possibly even a previous profile from the same patient to identify any biomarkers associated with disease, toxicity, or therapeutic enhancement.

As one skilled in the art may appreciate, an array of binding agents has a plurality of uses. Such uses include, but are not limited to, identification of cell-to-cell and molecular interactions, drug mode-of-action studies, cellular localization studies, investigation of molecular pathways, baseline determinations, drug toxicity studies, drug interaction studies, chemical inhibition analyses, metabolic profiling and the like.

As indicated herein, treatment of a disease state may be accomplished by administering an effective amount of a binding agent or epitope identified by the method of the present invention. A binding agent or epitope may be used or administered as a mixture, for example in equal amounts, or individually, provided in sequence, or administered all at once. In providing a patient with a binding agent or epitope, or fragments thereof, a binding agent or epitope is used in an amount effective to substantially alter or reduce, e.g., reduce by at least about 50%, the disease state or symptoms in the recipient.

To achieve the desired reductions, a binding agent or epitope may be administered in a variety of unit dosage forms. The dose will vary according to the particular binding agent. For example, different binding agents or epitopes may have different masses and/or affinities, and thus require different dosage levels.

Administration of a binding agent or epitope will generally be performed by an intravascular route, e.g., via intravenous infusion by injection. Other routes of administration may be used if desired. Formulations suitable for injection are found in Remington: The Science and Practice of Pharmacy, Alfonso R. Gennaro, editor, 20th ed. Lippincott Williams & Wilkins: Philadelphia, Pa., 2000. Such formulations must be sterile and non-pyrogenic, and generally will include a pharmaceutically effective carrier, such as saline, buffered (e.g., phosphate buffered) saline, Hank's solution, Ringer's solution, dextrose/saline, glucose solutions, and the like. The formulations may contain pharmaceutically acceptable auxiliary substances as required, such as, tonicity adjusting agents, wetting agents, bactericidal agents, preservatives, stabilizers, and the like.

As indicated, a binding agent identified by the method of the present invention may be used as delivery vehicles for drugs. For example, a cytotoxic drug may be covalently or noncovalently associated with a binding agent whose binding partner is a cell surface polypeptide only expressed in cells involved in the development of a disease state. The cytotoxic drug-binding agent combination would provide specific delivery of the cytotoxic drug to the cell of interest and minimize side effects associated with the delivery of said drug to other cell types.

A binding agent identified by the method of the present invention may also be used as an imaging marker. For example, a commonly used radiochemical such as Technicium may be covalently or noncovalently associated with a binding agent whose binding partner is a cell surface polypeptide only expressed in cells involved in the development of a disease state. The radiochemical-binding agent combination would provide for the clinical imaging, visualization and therefore detection of a disease state without the administration of large amounts of non-specific radiochemical and non-specific results. In this case only the disease state, such as a tumor, would be identified with a high level of confidence of the diagnosis.

Further, it is contemplated that an array of binding agents may be useful in plant breeding and quantitative and qualitative trait analyses. For example, a plant-derived epitope or binding agent or collection of plant epitopes or binding agents may be used as molecular markers for phylogenetic studies, characterizing genetic relationships among crop varieties, identifying crosses or somatic hybrids, and the study of quantitative inheritance. Moreover, disease resistance markers may be identified using the method of the invention.

What is claimed is:

1. A method for identifying a binding agent or epitope for use in drug design, drug targeting or diagnostics comprising the steps of:
contacting a collection of binding agents with a collection of epitopes so that a cognate binding agent and epitope bind;
sorting the bound binding agent and epitope from the collection;
characterizing the binding agent and epitope;
detecting the level or location of the characterized epitope in a sample using the characterized binding agent; and
correlating the level or location of the epitope in the sample with the presence or stage of a disease or condition so that a binding agent or epitope for use in drug design, drug targeting or diagnostics is identified.

2. The method of claim 1 further comprising the step of comparing the correlated level or location of the epitope in the sample with information in a database or publication.

3. The method of claim 1 wherein in the steps of contacting a collection of binding agents with a collection of epitopes so that a cognate binding agent and epitope bind and sorting the bound binding agent and epitope from the collection occur simultaneously.

4. A binding agent identified by the method of claim 1.

5. An epitope identified by the method of claim 1.
